(19) United States
(12) Patent Application Publication
(10) Pub. No.: US 2008/0208291 A1
(43) Pub. Date: Aug. 28, 2008

(54) FREQUENCY SHIFT KEYING (FSK) MAGNETIC TELEMETRY FOR IMPLANTABLE MEDICAL DEVICES AND ASSOCIATED SYSTEMS AND METHODS

(75) Inventors: Kent Leyde, Redmond, WA (US); Michael Bland, Seattle, WA (US)

Correspondence Address: PERKINS COIE LLP;PATENT-SEA, P.O. BOX 1247, SEATTLE, WA, 98111-1247, US

(73) Assignee: Northstar Neuroscience, Inc., Seattle, WA (US)

(21) Appl. No.: 11/923,553

(22) Filed: Oct. 24, 2007

Related U.S. Application Data
(60) Provisional application No. 60/854,322, filed on Oct. 24, 2006.

(51) Int. Cl. A61N 1/08

(52) U.S. Cl. 607/60

(57) ABSTRACT

A telemetry receive unit for use in an implantable medical device or system can include a coil unit, a single-bit analog to digital converter (ADC), a finite impulse response filter (FIR) coupled to the single-bit ADC, and an accumulator coupled to one or more filter taps of the FIR filter. The accumulator, in operation, can produce one or more data recognition signals corresponding to sampled bits at least temporarily stored at the FIR filter. In several examples, the data recognition signals discriminate between frequency modulated signals that are received at the coil unit, including frequency shift keying (FSK) modulated signals.

### CROSS-REFERENCE TO RELATED APPLICATION

This application claims priority to U.S. Provisional Patent Application No. 60/854,322, filed Oct. 24, 2006, which is hereby incorporated by reference.

### TECHNICAL FIELD

The present disclosure relates to systems and methods for magnetic telemetry. More particularly, the present disclosure describes various embodiments of FSK-based systems that facilitate communication between implanted medical devices and external programming, control, or communication devices.

### BACKGROUND

Implantable medical devices can facilitate the delivery of signals and/or substances to particular sites within the body, which can correspond to locations within or upon the brain, the spinal cord, peripheral nerves, muscles, glands, or other bodily tissues. Representative types of implantable medical devices include drug pumps, pacemakers, peripheral nerve stimulation devices, spinal column stimulation devices, cortical stimulation devices, and deep brain stimulation devices.

Typically, an external programmer transfers signals to or receives signals from an implanted medical device in accordance with a wireless signal transfer protocol, where such signals can correspond to programming instructions, implanted device operation parameters, patient physiologic signals, or other information. Implantable medical systems can be designed in view of prolonging the life or recharging interval associated with an implanted power source; providing an acceptable data communication rate between an implanted device and an external programmer; and/or achieving an acceptable communication distance or positional tolerance between an implanted device and an external programmer.

### BRIEF DESCRIPTION OF THE DRAWINGS

FIG. 1A is a schematic illustration of a medical treatment and/or monitoring system that includes an FSK-based magnetic telemetry unit according to an embodiment of the disclosure.

FIG. 1B is a schematic illustration of an implantable pulse generator (IPG) positioned within a patient's body.

FIG. 2 is an illustration of a representative symbol frame corresponding to the byte 01010011 in accordance with a set of FSK parameters suitable for particular embodiments of the disclosure.

FIG. 3A is a high-level block diagram of an FSK-based magnetic telemetry unit within a treatment delivery or other implanted device according to an embodiment of the disclosure.

FIG. 3B is a block diagram of a transmission unit according to one embodiment of the disclosure.

FIG. 3C is a block diagram of a reception unit according to an embodiment of the disclosure.

FIG. 4A is a schematic diagram of a reception unit according to particular embodiments of the disclosure.

FIGS. 4B and 4C are schematic diagrams of a first and a second FIR filters according to a representative embodiment of the disclosure.

FIGS. 5A and 5B are schematic illustrations of the first and second FIR filters relative to representative sampled mark and space waveform values.

FIGS. 6A and 6B are tables listing a first and a second set of coefficients corresponding to a first and a second set of filter taps.

FIG. 6C is a graph illustrating digital mark and space waveforms generated in accordance with a representative embodiment of the disclosure.

### DETAILED DESCRIPTION

### A. Introduction

The following disclosure describes various embodiments of Frequency Shift Keying (FSK) based systems, apparatus, circuits, methods, and procedures for providing power efficient, noise tolerant, and positionally tolerant wireless signal transfer between devices configured to communicate through magnetic telemetry. In general, such devices can form portions of a medical treatment and/or monitoring system.

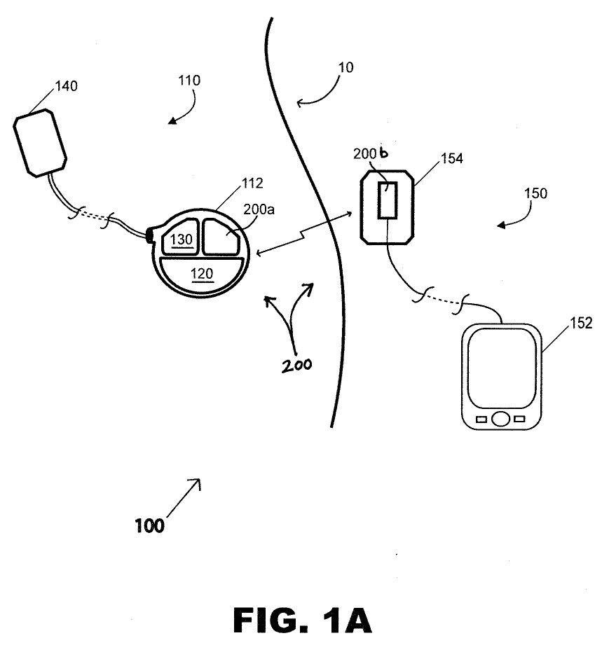

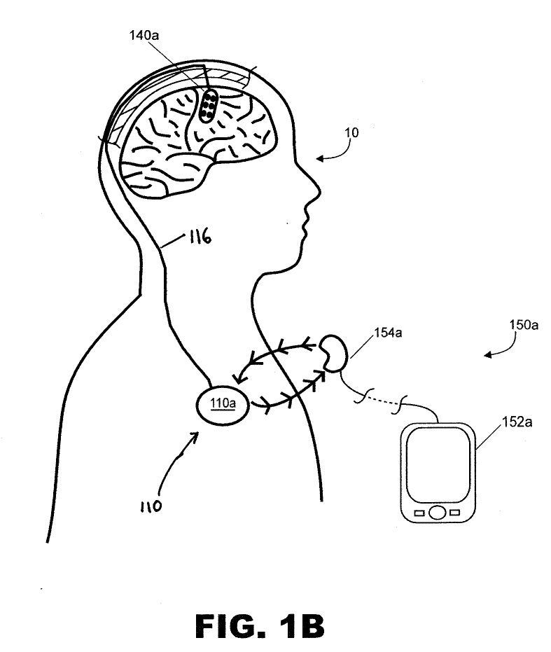

FIG. 1A is a schematic illustration of a medical treatment and/or monitoring system 100 that includes an FSK-based magnetic telemetry unit 200 according to an embodiment of the disclosure. In one embodiment, the system 100 includes at least one implanted device 110 that resides within an individual's body 10 (e.g., at a subcutaneous location within or proximate to the torso, the skull, the spinal column, a limb, or other anatomical region), and at least one communication device 150 that is external to the body 10.

In general, an implanted device 110 can include essentially any type of implantable device or device set that includes an FSK-based magnetic telemetry unit 200a configured for signal transfer with the external communication device 150. The external communication device 150 can include essentially any type of programmable or programmed device (e.g., a personal digital assistant (PDA) 152 or other type of control unit) having or coupled to a signal exchange module 154 (e.g., a programming wand or puck). The signal exchange module 154 includes an FSK-based magnetic telemetry unit 200b that is functionally complementary to the telemetry unit 200a within the implanted device 110. Each FSK-based magnetic telemetry unit 200a, 200b can send and/or receive signals to facilitate, for example, the monitoring, interrogation, control, operation, and/or programming of the implanted device 110 by the external communication device 150.

In several embodiments, the implanted device 110 can include a treatment delivery device having a power source 120; a therapy unit 130; a set of signal and/or substance transfer elements 140; and the FSK-based magnetic telemetry unit 200a. The treatment delivery device 110 further includes at least one housing 112 that provides a biocompatible hermetically sealed barrier between treatment device elements and the body 10. The power source 120 can include a battery and/or a capacitor, and in some embodiments can be rechargeable or replenishable. The therapy unit 130 can include one or more of a processing unit (e.g., an Application Specific Integrated Circuit (ASIC)); a memory or other type of electronically readable or programmable medium; a signal generation, delivery, or sensing device (e.g., an electrical, magnetic, optical, thermal, or other signal generation or transfer device); a substance delivery or sensing device (e.g., a drug or other chemical substance infusion device); and/or another type of device. The therapy unit 130 is coupled to the set of signal and/or substance transfer elements 140 in a manner that facilitates the provision of one or more treatments or therapies to which the implanted device 110 is directed.

In a representative embodiment, the implanted device 110 can include a pulse generator that is coupled to a set of electrodes. FIG. 1B is a schematic illustration of a representative implantable pulse generator (IPG) 110a positioned within a patient's body 10. A lead wire 116 can couple the IPG 110a to one or more electrode assemblies 140a that are implanted relative to a set of stimulation sites. While the electrode assembly 140a shown in FIG. 1B can be configured to provide epidural or subdural cortical stimulation, different or additional types of electrode assemblies can be coupled to the IPG 110a to provide deep brain, spinal column, peripheral nerve, vascular structure, or other types of stimulation. Depending upon embodiment details, the implanted device 110 can also include patient monitoring hardware and/or software (e.g., electrocorticography (ECoG) circuitry). In another representative embodiment, the implanted device 110 can additionally or alternatively include one or more chemical substance transfer devices, for example, a drug infusion pump.

### B. FSK Signaling Aspects of Various Embodiments

In an FSK system, a digital value of 1 is represented by an analog waveform or “symbol” corresponding to a first frequency, and a digital value of 0 is represented by an analog waveform or “symbol” corresponding to a second frequency. The analog waveform corresponding to the digital value of 1 is commonly referred to as a “mark” symbol, and that corresponding to the digital value of 0 is commonly referred to as a “space” symbol. The absence of a mark or a space is typically defined to be an “idle” symbol. Sequences of symbols are organized into and transferred as signal “frames.” Any given frame includes a predetermined start symbol sequence, followed by a predetermined number of mark and/or space symbols (e.g., 8 mark and/or space symbols, which would represent 1 byte of data), followed by a predetermined stop symbol sequence (which can include one or more idle symbols). Symbols can be defined to have an equal, predetermined temporal duration or “bit width,” such that a mark or a space spans an integral number of waveform periods or cycles.

In various embodiments in accordance with the present disclosure, the symbol duration and mark and space frequencies can be defined or chosen such that signal modulation, demodulation, and transfer operations result in a) an acceptable data transfer rate (i.e., bit rate or baud rate); b) signals that are readily distinguishable, and which exhibit minimal frequency harmonic overlap; c) acceptable levels of power consumption; and/or d) a reduced or low likelihood of signal interference or corruption in the presence of potential interference sources. Potential interference sources can include equipment or devices that are present in an environment such as a medical office, a home, or other setting. Representative types of interference sources can include computer equipment (e.g., CRT monitors), appliances, various types of motors, and other devices.

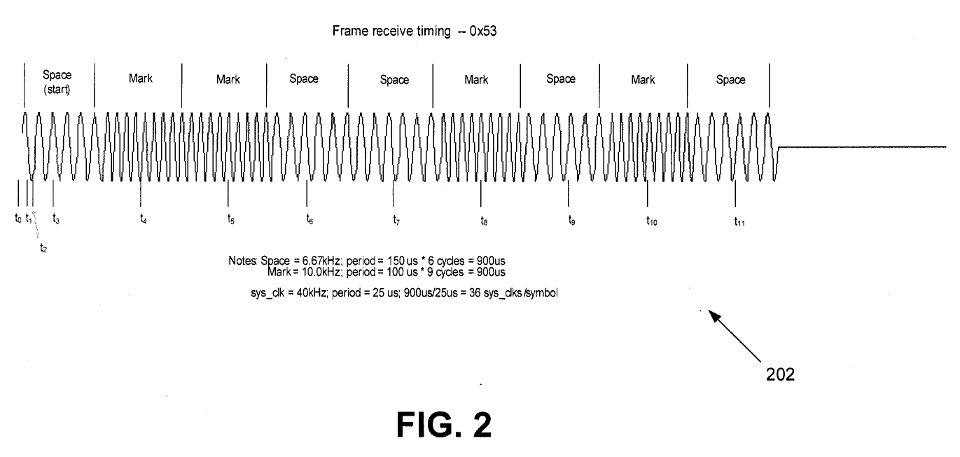

In a representative embodiment, the symbol duration can equal or approximately equal 900 microseconds; a mark frequency can equal or approximately equal 10 kHz; and a space frequency can equal or approximately equal 6.67 kHz. In such an embodiment, 9 waveform cycles at 10 kHz form a mark, and 6 waveform cycles at 6.67 kHz form a space. The baud rate, which equals the reciprocal of the symbol duration, equals 1111.11 in this embodiment. FIG. 2 is an illustration of a representative symbol frame 202 defined in accordance with the above FSK parameters. The representative symbol frame 202 encodes the byte 01010011 (in reverse bit order), and includes a space as its start symbol and two idle symbols as its termination sequence. Those skilled in the relevant art will understand that different embodiments can employ other symbol durations, mark frequencies, space frequencies, and/or symbol frame encoding schemes.

In general, the implanted device 110 (FIG. 1A) includes a limited lifetime or limited capacity power source such as a battery and/or a capacitor. Hence, the implanted device 110 is typically subject to more stringent power limitations than the external communication device 150 (FIG. 1A). As a result, appropriately reducing and/or managing power consumption by the implanted device 110 can prolong or significantly increase the power source lifetime or recharging interval. Relative to telemetry operations, in several situations the implanted device 110 can be expected to receive signals from the external communication device 150 more often than it sends signals to the external communication device 150. Power can therefore be conserved through a telemetry system design that incorporates low complexity, computationally efficient signal reception circuitry that operates at a low to moderate clock frequency, as described in detail hereafter.

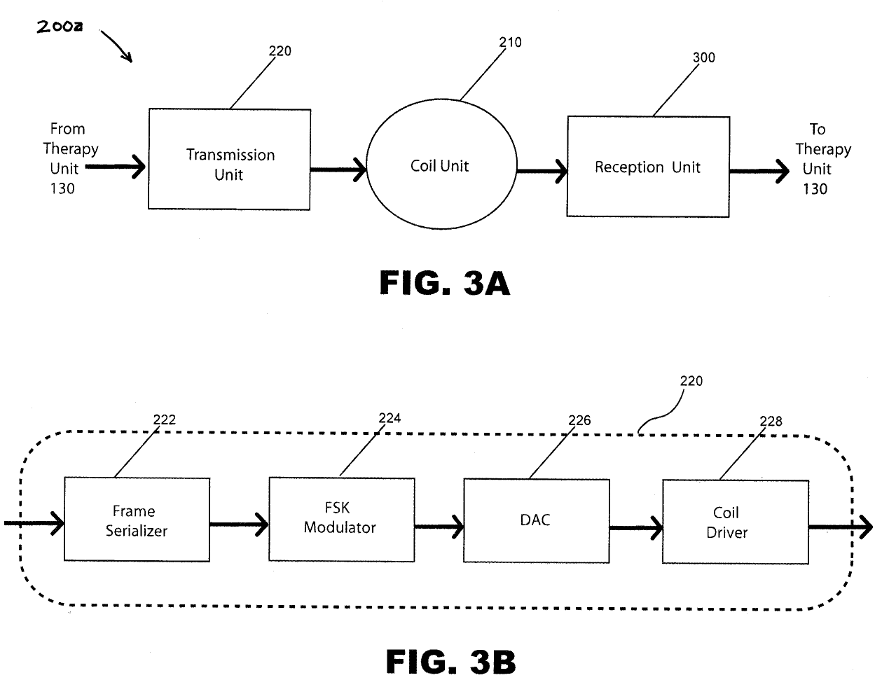

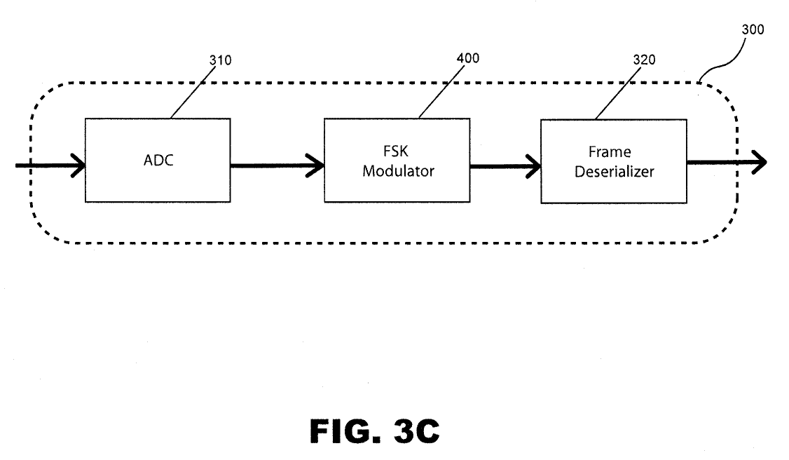

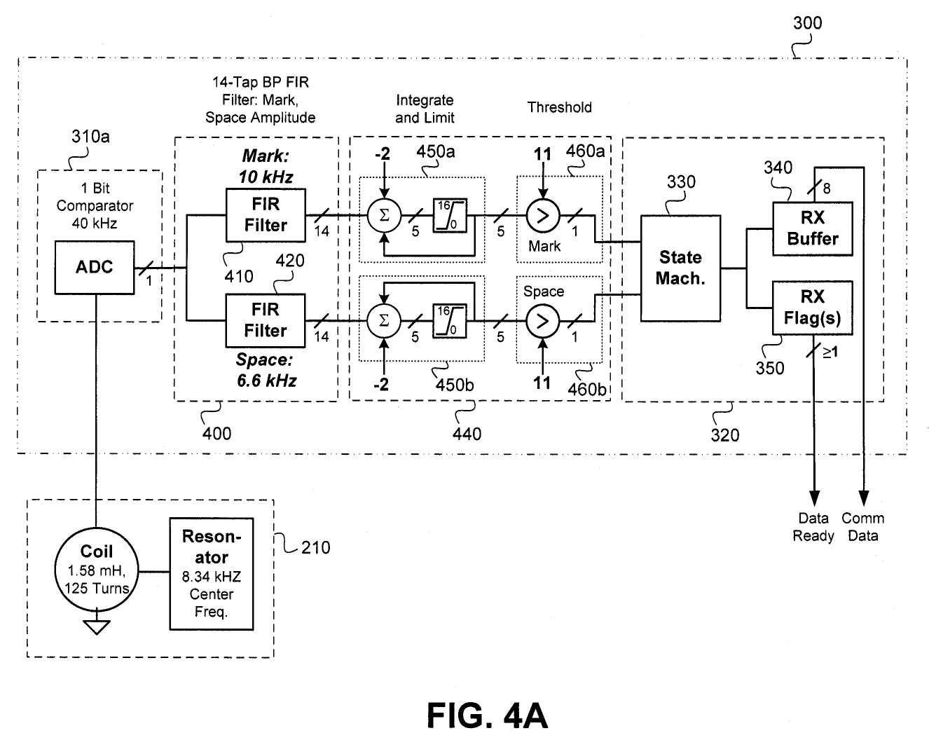

FIG. 3A is a high-level block diagram of an FSK-based magnetic telemetry unit 200a within a treatment delivery or other implanted device 110 according to an embodiment of the disclosure. In general, the telemetry unit 200a can include a coil unit 210, a transmission unit 220, and a reception unit 300. The coil unit 210 can include a resonator (e.g., a crystal oscillator) coupled to a wire coil or loop that is sized, shaped, and compositionally constructed in a manner that facilitates the generation of an electrical signal in the presence of a time varying magnetic signal, and vice versa, in a manner readily understood by those skilled in the relevant art.

Referring now to FIG. 3A together with FIGS. 1A and 1B, in response to commands and/or data received from the therapy unit 130, the transmission unit 220 performs signal restructuring, signal conversion, and/or signal conditioning operations, and controls or drives the coil unit 210 to generate a time varying magnetic signal. The time varying magnetic signal radiates or propagates through space, and can be detected by the external communication device 150. FIG. 3B is a block diagram of the transmission unit 220 according to one embodiment of the disclosure. The transmission unit 220 can include, for example, a frame serializer 222 that is coupled to receive and sequence digital signals from the therapy unit 130; an FSK modulator 224; a Digital to Analog Converter (DAC) 226; and a coil driver 228.

In response to the presence of appropriately oriented time varying magnetic signals, the coil unit 210 (FIG. 3A) generates corresponding electrical signals. The reception unit 300 (FIG. 3A) receives such electrical signals, and performs signal conditioning, signal conversion, and/or signal structuring operations. The reception unit 300 can store appropriately structured signals, and/or transfer them to the therapy unit 130 (FIG. 1A). FIG. 3C is a block diagram of the reception unit 300 according to an embodiment of the disclosure. The reception unit 300 can include, for example, an Analog to Digital Converter (ADC) 310 that is coupled to an FSK demodulator 400, which is coupled to a frame deserializer 320.

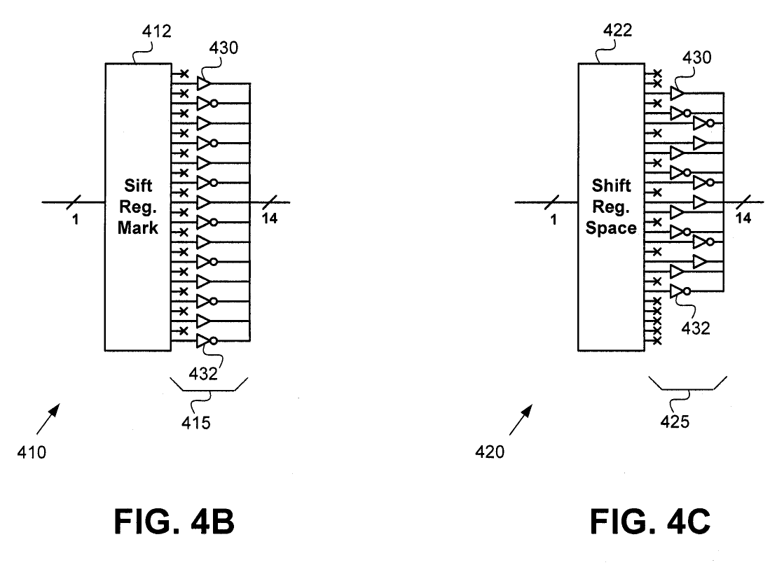

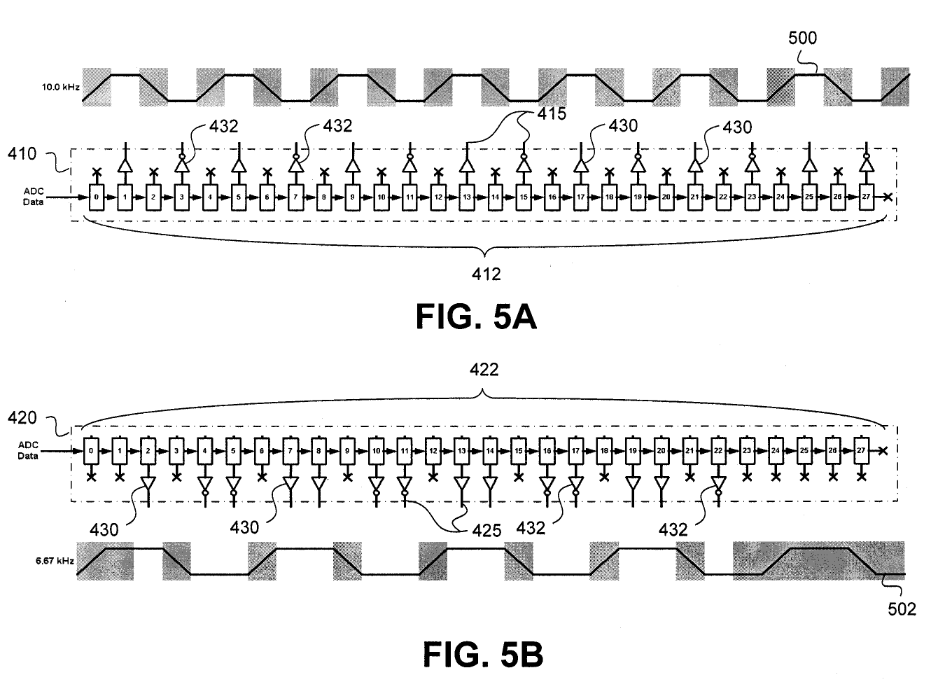

FIG. 4A is a schematic diagram of a reception unit 300 according to particular embodiments of the disclosure. In one embodiment, analog to digital conversion can be performed by a single-bit ADC 310a; the FSK demodulator 400 can include a signal filter; and the frame deserializer 320 can include a state machine 330. In a representative embodiment, the single-bit ADC 310a can be implemented using a single-bit comparator. Implementation of an ADC 310 using a single-bit comparator 310a rather than a conventional type of multiply-add ADC structure results in simplified circuitry and reduced power consumption. The FSK demodulator 400 can be implemented using first and second Finite Impulse Response (FIR) filters 410, 420, which are coupled to a thresholding accumulator 440. Further, the frame deserializer 320 can be implemented using a state machine 330 that is coupled to a buffer 340 and a notification unit 350.

FIGS. 4B and 4C are schematic diagrams of the first and second FIR filters 410, 420 according to a representative embodiment of the disclosure. In this embodiment, the first FIR filter 410 includes a first shift register 412 coupled to a first set of filter taps 415, and the second FIR filter 420 includes a second shift register 422 coupled to a second set of filter taps 425. As described in detail hereafter, the structure of the FIR filters 410, 420 facilitates the performance or execution of a relative comparison, matching, or scoring operation between 1) a series of 1-bit values output by the ADC converter 310a, where such values correspond to signal values within an analog waveform generated by the coil unit 210 as a result of magnetic induction; and 2) a pair of reference digital waveforms, each of which corresponds to a digital model of a mark or a space symbol oscillation pattern. Those of ordinary skill in the relevant art will understand that in various embodiments, the structures of the first and second FIR filters 410, 420 correspond or generally correspond to a matched filter design. Further, those of ordinary skill in the art will also appreciate that the first and second FIR filters 410, 420 can be combined or aggregated into a single FIR filter. For example, such a filter can include a shift register having multiple outputs (or filter taps) that are coupled to an arrangement of inverters and buffers that yield substantially the same output of the two separate FIR filters 410, 420.

Reception unit elements operate in accordance with a clock signal generated within the implanted device 110. The reception unit clock frequency should be sufficiently high to facilitate accurate signal recovery (or an adequate likelihood of signal recovery), yet sufficiently low to avoid unnecessary power consumption. In the following discussion, various elements within the reception unit 300 can operate at a clock frequency of approximately 40 kHz. In addition, mark and space symbol frequencies are respectively defined to approximately equal 10 kHz and 6.67 kHz, and symbol durations are defined to approximately equal 900 microseconds. Those skilled in the relevant art will understand that in different embodiments, reception unit elements can operate at other frequencies, and/or symbol oscillation frequencies or symbol durations can be different.

The single-bit comparator 310a (FIG. 4A) samples the signal generated by the coil unit 210 (FIG. 3A), and outputs a 1-bit sampled signal value (which can correspond to a binary value of 1 or 0) to the first and second shift registers 412, 422. Since the comparator 310a operates at 40 kHz in the representative embodiment under consideration, 1-bit sampled values are generated every 25 microseconds. As described above, the mark and space symbol durations can be defined to be approximately 900 microseconds. Sampling each 900 microsecond mark or space symbol at 40 kHz could yield a total of 36 digital samples per mark or space symbol (i.e., a 900 microsecond symbol duration divided by 25 microseconds per sample gives a total of 36 possible samples). However, in several embodiments, in order to reduce a likelihood of inter-symbol interference or overlap, the FIR filters 410, 420 can receive and operate upon fewer than the total number of possible samples available Sₐ per symbol. Thus, the total bit width W of the FIR filters 410, 420 can span a smaller number of bits than Sₐ, the total number of potentially available samples (which equals 36 bits in the embodiment under consideration). In a representative embodiment, the total bit width W of the FIR filters 410, 420 can equal 28 bits, which is sufficient to provide reliable identification of mark or space symbols. In addition to reducing a likelihood of inter-symbol interference, a shortened or truncated FIR filter implementation can reduce circuit complexity, which can further reduce power consumption.

In the embodiment under consideration, the FIR filters 410, 420 also operate at 40 kHz. Hence, single bit values output by the comparator 310a are serially clocked into the FIR filter shift register structures every 25 microseconds. Similarly, bits within the FIR filter shift register structures corresponding to digital waveform values are sequentially clocked out of the FIR filter shift register structures every 25 microseconds. The first and second sets of filter taps 415, 425 include buffers 430 and inverters 432 that operate upon particular shift register outputs. The buffers 430 and inverters 432 mathematically operate upon the shift register outputs to which they are coupled. Mathematically, the buffers 430 perform an identity or input-to-output signal preservation operation, and the inverters 432 perform a binary logic inversion operation. Those skilled in the relevant art will understand that when treating the shift register contents as a waveform having peak positive and negative amplitudes centered about an average amplitude of zero, a buffer 430 corresponds to a signal multiplier of 1; an inverter corresponds to a signal multiplier of −1; and the absence of a buffer 430 or an inverter 432 (an open circuit, a “no coupling,” or a “no connection” condition) corresponds to a signal multiplier of 0.

In general, the multiplier values corresponding to the first and second sets of filter taps 415, 425 can be viewed as forming 1) a predetermined digitized mark symbol reference oscillation or value transition pattern; and 2) a predetermined digitized space symbol reference oscillation or value transition pattern, against which the FIR filter shift register contents at any given time are correlated, and subsequently operated upon by the thresholding accumulator 440, as further described below.

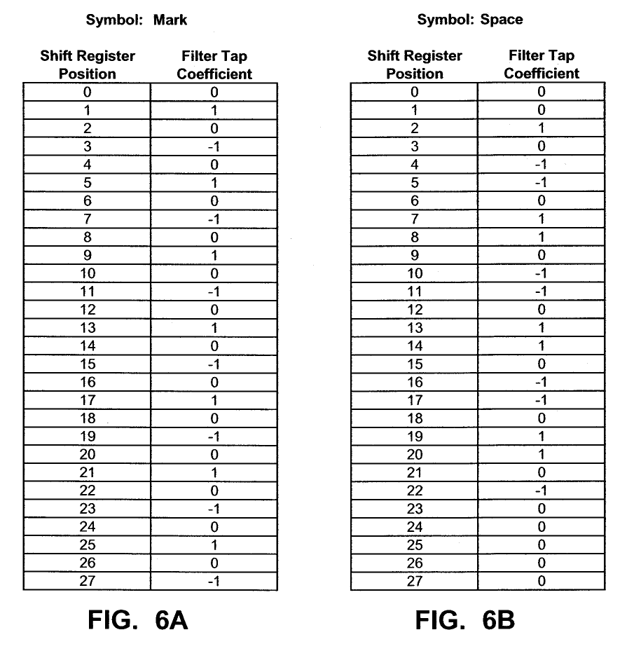

FIGS. 5A and 5B are schematic illustrations of the first and second FIR filters 410, 420 relative to representative sampled mark and space waveform values. In the illustrated representative embodiment, each of the first and second FIR filters 410, 420 can be implemented using a single 28-bit shift register 412, 422 into which W=28 1-bit values corresponding to the analog waveform sampled by the 1-bit comparator 310a (FIG. 4A) can be shifted. In FIG. 5A, an upper digital waveform 500 illustrates a representative sequence of sampled values corresponding to a 10 kHz mark symbol; and in FIG. 5B, a lower digital waveform 502 illustrates a representative sequence of sampled values corresponding to a 6.67 kHz space symbol.

The buffers 430 and inverters 432 within the first set of filter taps 415 are shown aligned relative to a series of representative mark symbol samples that could have been shifted into the first FIR filter 410 during a first time interval; and the buffers 430 and inverters 432 within the second set of filter taps 425 are shown aligned relative to a series of representative space symbol samples that could have been shifted into the second FIR filter 420 during a second time interval. To aid understanding, the high and low values corresponding to the 10 kHz waveform are shown as having an in-phase alignment with the first set of filter taps 415; and the high and low values corresponding to the 6.67 kHz waveform are shown as having an in-phase alignment with the second set of filter taps 425.

With appropriate sets of filter taps 415, 425 that define corresponding appropriate sets of multiplier values, a sequence of stored shift register values corresponding to an error-free, in-phase waveform can result in each of the filter taps outputting a digital value of 1, giving a highest measure or level of correlation with a mark or a space signal. Moreover, for a simplified circuit design (and hence lower power consumption), stored shift register values corresponding to high-to-low and/or low-to-high sampled waveform transitions within the in-phase waveform can be ignored (or treated as “don't care” conditions with respect to filter tap multiplier values), essentially without affecting a correlation measure.

In particular, for an uncorrupted, error-free, or acceptably error-free waveform, when stored shift register samples having a value of 1 are in-phase with the buffers 430, and stored shift register samples having a value of 0 are in-phase with the inverters 432, each buffer 430 and each inverter 432 outputs a binary 1. This can be defined as a highest or maximal degree of in-phase correlation. Analogously, when stored shift register samples having a value of 1 are 180 degrees out of phase with buffers 430 (and therefore in-phase with inverters 432), and stored shift register samples having a value of 0 are out of phase with inverters 432 (and therefore in-phase with buffers 430), each buffer 430 and each inverter 432 outputs a binary 0. This can be defined as a highest or maximal degree of out-of-phase correlation.

In a representative 28-bit FIR filter embodiment, when shift register samples of an acceptably error-free 10 kHz waveform in which values corresponding to binary 1 are in-phase with buffers 430 and values corresponding to binary 0 are in-phase with inverters 432, a number of buffers 430 and inverters 432 within the first set of filter taps 415 that provides a highest measure of in-phase correlation between filter multiplier values and the values of waveform samples in the shift register can include 7 buffers 430 and 7 inverters 432. In this embodiment, the first set of filter taps 415 provides a first subset of FIR filter outputs spanning 14 bits. For acceptably error-free in-phase samples, each of the 14 outputs of the first set of filter taps 415 corresponds to a binary value of 1. In an analogous manner, for acceptably error-free out-of-phase samples, each of the 14 outputs of the first set of filter taps 415 corresponds to a binary value of 0. As will be described in more detail below, the bit values of individual filter taps 415 are summed at the first integration unit 450a (FIG. 4A).

Similarly, for acceptably error-free in-phase samples of a 6.67 kHz waveform, a number of buffers 430 and inverters 432 within the second set of filter taps 425 that provides a highest measure of in-phase correlation between filter tap multiplier values and shift register contents can include 7 buffers 430 and 7 inverters 432. The second set of filter taps 425 therefore provides a second subset of FIR filter outputs, also spanning 14 bits. For in-phase samples of the 6.67 kHz waveform, each such output has a binary value of 1; and for out-of-phase samples, each such output has a binary value of 0. As will be described in more detail below, the bit values of individual filter taps 425 are summed at the second integration unit 450b (FIG. 4A).

In general, the number and organization of buffers 430 and inverters 432 within each of the first and second sets of filter taps 415, 425 can be defined in accordance with the following equation:

FilterCoeff(N, NumSamples, NumCycles)=Round(1.4((sin(N/NumSamples)*NumCycles*2*pi))) Eq. 1

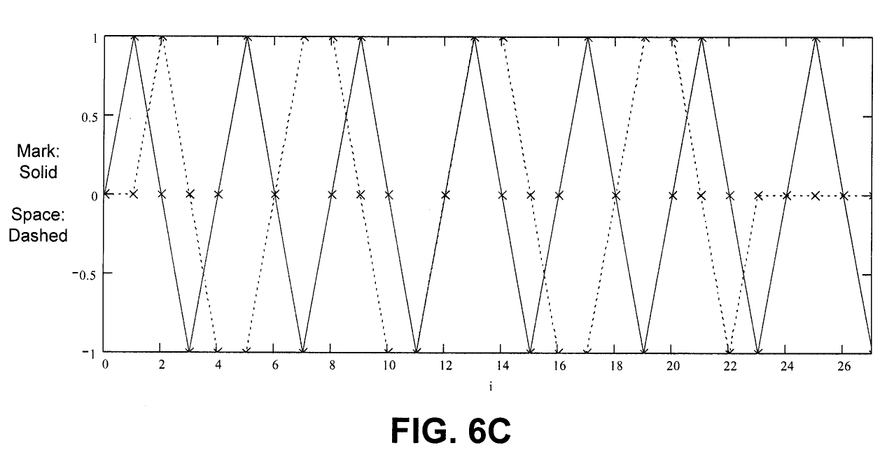

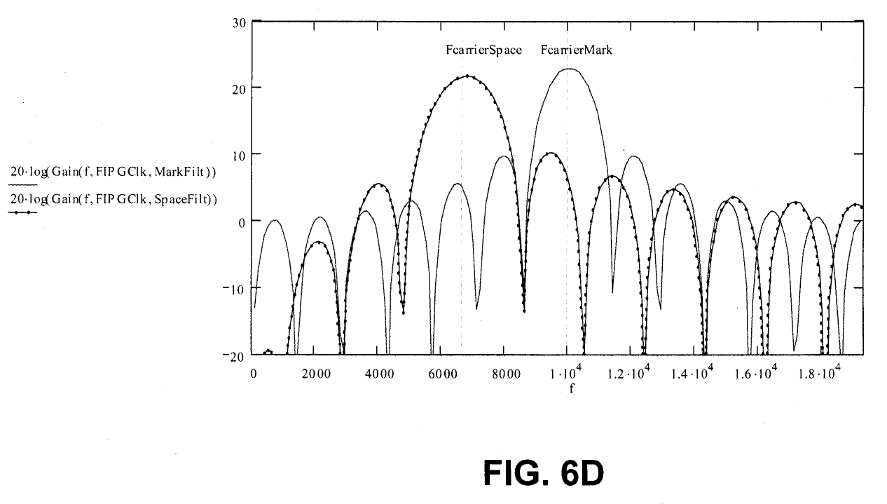

FIGS. 6A and 6B are tables listing the coefficients for the first and second sets of filter taps 415, 425, respectively, in accordance with Equation 1. Within the first set of filter taps 415, the buffers 430 and the inverters 432 can be positioned to receive particular shift register outputs in the manner shown in FIG. 5, such that the buffers 430 are sequentially separated from inverters 432 by a shift register output that lacks a coupling or a connection to either a buffer 430 or an inverter 432. Within the second set of filter taps 425, the buffers 430 and the inverters 432 are positioned in the generally pairwise manner shown in FIG. 5.

FIG. 6C is a graph illustrating digital mark and space waveforms generated in accordance with Equation 1. FIG. 6D is a graph illustrating a frequency response curve of the first and second FIR filters 410, 420 designed in accordance with Equation 1. As indicated by FIG. 6D, in this embodiment the FIR filters 410, 420 provide good discrimination between the 10 kHz and 6.67 kHz mark and space center frequencies, as well as a good or an adequate level of attenuation between such center frequencies and the associated sideband frequencies.

As further described below, the thresholding accumulator 440 (FIG. 4A) treats or interprets the bits spanned by the outputs of the first set of filter taps 415 as representative of a single number, which is referred to herein as a mark correlation value. A mark correlation value corresponds to a value of 14 for a highest or strongest degree of in-phase correlation with a mark symbol, and a value of 0 for a highest degree of out-of-phase correlation with a mark symbol. Analogously, the thresholding accumulator 440 treats or interprets bits spanned by the outputs of the second set of filter taps 425 as a single number, which is referred to herein as a space correlation value. A space correlation value has a value of 14 for a highest in-phase correlation condition with a space symbol and a value of 0 for a highest out-of-phase correlation condition with a space symbol.

The signal generated by the implanted device's coil unit 210 varies in accordance with the particular sequence(s) of marks and spaces encoded within a transmission received from the external programming device 150. Hence, the values stored within the FIR filter's shift register(s) will change with time as sampled ADC values are progressively shifted in. Because values output by the 1-bit ADC 310a are successively shifted in at a rate corresponding to ADC sampling frequency, a signal that is 180 degrees out-of-phase at a particular time with respect to a set of filter taps 415, 425 can become in-phase or more in-phase as one or more subsequent values output by the ADC 310a are shifted in. Analogous considerations apply to the shift register contents in general, that is, in-phase shift register contents can shift into an out-of-phase state relative to the first and second sets of filter taps 415, 425 as a result of the successive receipt of new ADC output values.

Those of ordinary skill in the relevant art will understand that in normal operating environments, at any given time the strength and/or quality of the signal output by the coil unit 210 can depend upon 1) the presence of interference sources; as well as 2) the distance between and/or relative positions and orientations of the coil unit 210a within the implanted device 110 and a coil unit 210b within the external programming device 150. Therefore, sampled waveforms can include errors or nonideal characteristics (e.g., as a result of interference during signal transmission, or a transmission-to-reception distance that exceeds a distance associated with a reliable signal recovery likelihood). At any given time, the sampled waveform values within the FIR filters 410, 420 can exhibit amplitude and/or phase relationships that give rise to a mark correlation value or a space correlation value between 0 and 14. A mark correlation value or a space correlation value of 7 can be interpreted as indicating a lowest degree of in-phase or out-of-phase correlation with either a mark or a space symbol.

Referring now to FIGS. 4A-4C, the thresholding accumulator 440 is coupled to receive mark and space correlation values from the first and second sets of output taps 415, 425. In one embodiment, the thresholding accumulator 440 includes a first integration unit 450a coupled to a first threshold comparator 460a; and a second integration unit 460a coupled to a second threshold comparator 460b. The first integration unit 450a is coupled to receive the mark correlation value from the first set of filter taps 415, and the second integration unit is coupled to receive the space correlation value from the second set of filter taps 425.

In particular embodiments, the first and second integration units 450a, 450b each include a relative magnitude leaky integrator, further details of which are described hereafter in the context of the first integration unit 450a. Those of ordinary skill in the relevant art will understand that identical, essentially identical, or analogous considerations apply to the second integration unit 450b. The first integration unit 450a receives a mark correlation value from the first FIR filter 410, and treats the mark correlation value as an absolute value, an offset, or a relative magnitude with respect to a reference average or median value. In various embodiments, the reference average value corresponds to a lowest degree of in-phase or out-of-phase correlation with a mark symbol, that is, a mark correlation value of 7.

The first integration unit 450a 1) summates or integrates an extent to which received mark correlation values deviate from the reference average value, and 2) outputs a present mark integration value. In various embodiments, the first integration unit 450a limits the present mark integration value in accordance with a maximum integration value. In a representative embodiment, the maximum integration value equals 16.

The first integration unit 450a incorporates a subtraction operation into the aforementioned summation or integration, which causes the present mark integration value to decay toward zero over time in the event that received mark correlation values exhibit little or no deviation from the reference average value. Those of ordinary skill in the relevant art will understand that this subtraction operation facilitates a “leaky” integration. In a representative embodiment, the subtraction operation is performed in accordance with a decrement value of −2.

The first threshold comparator 460a is coupled to receive the present mark integration value output by the first integration unit 450a, and compare the present mark integration value to a mark symbol threshold value, which in a representative embodiment equals 11. The first threshold comparator 460a can further generate a mark recognition signal. In the event that the present mark integration value exceeds the mark symbol threshold value, the first threshold comparator 460a outputs a mark recognition signal having a value of 1; otherwise, the first threshold comparator 460a outputs a 0.

Similar considerations to those described above can apply to the second integration unit 450b and the second threshold comparator 460b. The second integration unit 450b can generate a present space integration value (typically in accordance with a leaky integration), and the second threshold comparator 460b outputs space recognition signal respectively having a value of 1 or 0 in the event that the present space integration value (which can be limited to a maximum integration value, e.g., 16) is greater or less than a space symbol threshold value (e.g., 11).

Referring again to FIG. 4A, in various embodiments the frame deserializer 320 can include a state machine 330, a receive buffer 340, and a flag unit 350. Those of ordinary skill in the relevant art will understand that in some embodiments, one or more portions of the frame deserializer 320 can structurally or functionally correspond to a Universal Asynchronous Receiver/Transmitter (UART). In one embodiment, the state machine 330 is coupled to receive the mark and space recognition signals output by the thresholding accumulator 440. In general, the state machine 330 can include hardware and/or software configured to interpret the mark and space recognition signals in a mutually exclusive manner to determine whether a mark symbol or a space symbol has been successfully received. More particularly, at any given time, in the event that the mark recognition signal indicates the presence of a mark symbol and the space recognition signal indicates the absence of a space symbol, the state machine 330 can define a bit within a data parcel currently under construction (e.g., a nibble, a byte, or a word) as a 1, at an appropriate sequential position or location relative to a most-recently defined bit within the data parcel. Similarly, in the event that the space recognition signal indicates the presence of a space symbol and the mark recognition signal indicates the absence of a mark symbol, the state machine 330 can define an appropriate bit within a data parcel as a 0.

In the event that each of the mark and space recognition signals simultaneously or essentially simultaneously indicate that both a mark and a space symbol have been received, the state machine 330 can issue an error signal, a frame invalid signal, and/or other type of signal to the flag unit 350. In certain embodiments, in response to such a condition, the state machine 330 can discard the data parcel currently under consideration. In the event that neither of the mark and space recognition symbols indicate that neither a mark nor a space symbol have been received, the state machine 330 can issue an awaiting data signal, a data ready signal, and/or another signal to the flag unit 350.

In various embodiments, the state machine 330 controls the loading and clearing of the receive buffer 340. The state machine 330 can, for example, issue 1 and/or 0 values to the receive buffer in order to construct a present data parcel within the receive buffer 340 itself. The state machine 330 can alternatively include one or more internal buffers or other data storage devices to facilitate the construction of a data parcel within the state machine 330 itself, after which the state machine 330 can transfer an entire data parcel to the receive buffer 340. In some embodiments, the receive buffer 340 can include a First-in, First-out (FIFO) buffer, in a manner understood by those skilled in the art. In certain embodiments.

Referring again to FIG. 1A, based upon one or more signals output by the flag unit 350, a therapy unit 130 can retrieve and subsequently process a set of data parcels stored within the receive buffer 340. In some embodiments, one or more portions of a therapy unit 130 can remain in an idle, inactive, or hibernate state for a given period of time, and periodically (e.g., once every k seconds, once every q minutes, or in accordance with some other time interval) transition to an active or awake state to determine whether the flag unit 350 indicates that data is ready for retrieval or processing. Alternatively, one or more portions of a therapy unit 130 can remain in an idle, inactive, or hibernate state until a signal output by the flag unit 350 automatically triggers a therapy unit state transition. A therapy unit idle, inactive, or hibernate state can facilitate reduced therapy unit power consumption.

One feature of at least some of the foregoing embodiments is that single-bit ADCs have fewer components than multi-bit ADCs and thus consume less power. Further, to process the digital signals of the ADC, circuits downstream from the single-bit ADC can use less complicated components. For example, downstream components of a multi-bit ADC need additional (power consuming) input/outputs and related logic to process multi-bit signals. Accordingly, embodiments of implanted devices that include the foregoing single-bit ADCs can have a reduced battery or capacitor size relative to conventional (telemetric) implanted devices. Additionally or alternatively, embodiments of the implanted device can also have a longer battery or capacitor life than conventional (telemetric) implanted devices.

From the foregoing, it will be appreciated that specific embodiments of the disclosure have been described herein for purposes of illustration, but that various modifications can be made without deviating from the disclosure. Additionally, certain aspects of the disclosure described in the context of particular embodiments can be combined, eliminated, or differently organized in other embodiments. For example, one or more structural or functional aspects of a receive unit 300 within or coupled to an implanted device 110 can additionally or alternatively exist within a receive unit within or coupled to an external programming device 150. Further, while advantages associated with certain embodiments of the disclosure have been described in the context of those embodiments, other embodiments can also exhibit such advantages, and not all embodiments need necessarily exhibit such advantages.

I/We claim:

1. A telemetry receive unit, comprising:
a coil unit;
a single-bit analog to digital converter (ADC) having an analog input coupled to the coil unit;
a finite impulse response filter (FIR) coupled to a digital output of the single-bit ADC; and
an accumulator that produces one or more data recognition signals corresponding to sampled bits at least temporarily stored at the FIR filter, the accumulator being coupled to one or more filter taps of the FIR filter.

2. The telemetry receive unit of claim 1 wherein the data recognition signals discriminate between first and second frequency modulated signals that are received at the coil unit.

3. The telemetry receive unit of claim 1 wherein the single-bit ADC includes a comparator that samples frequency shift keying (FSK) modulated signals at the coil unit, and wherein the data recognition signals of the accumulator include FSK mark and/or space recognition signals.

4. The telemetry receive unit of claim 1 wherein the FIR filter comprises:
a first shift register that produces a multi-bit mark value corresponding to a frequency shift keying (FSK) signal at the coil unit; and
a second shift register that produces a multi-bit space value corresponding to the FSK signal.

5. The telemetry receive unit of claim 1 wherein the accumulator includes one or more leaky integrators that sum multi-bit mark and/or space values produced at the filter taps of the FIR filter.

6. The telemetry receive unit of claim 1, further comprising a state machine that receives the data recognition signals.

7. The telemetry receive unit of claim 1 wherein the coil unit, the single-bit ADC, the FIR filter, and the accumulator are at least partially disposed within a housing that is implantable within an individual's body.

8. A patient-implantable device, comprising:
a housing that is implantable within an individual's body; and
a telemetry receive unit at least partially disposed within the housing and including:
a coil unit that generates an electrical signal in the presence of a time varying magnetic signal;
an analog to digital converter (ADC) that samples the electrical signal at periodic intervals and outputs only one bit of sampled data at individual periodic intervals; and
a sample evaluation unit that receives the sampled electrical signal.

9. The device of claim 7 wherein the time varying magnetic signal is produced, at least in part, by a communication device not disposed within the individual's body.

10. The device of claim 7 wherein the time varying magnetic signal is a modulated frequency shift keying signal (FSK).

11. The device of claim 7, further comprising one or more shift registers that receive the sampled data at the individual periodic intervals.

12. The device of claim 7, further comprising a frequency impulse response (FIR) filter coupled to the ADC, the FIR filter having filter taps that are selectively coupled to individual buffers and/or individual inverters, the individual buffers and/or inverters being arranged to produce multi-bit mark and/or multi-bit space values corresponding to the electrical signal.

13. The device of claim 7 wherein the sample evaluation unit includes:
a frequency impulse response (FIR) filter coupled to the ADC, the FIR having a shift register that stores individual sampled bits; and
at least one leaky integrator that sums the individual sampled bits of data to produce, at least in part, one or more data recognition signals.

14. The device of claim 7 wherein the sample evaluation includes:
a frequency impulse response filter that produces multi-bit mark and/or multi-bit space values corresponding to a sampled frequency shift keying (FSK) signal at the ADC; and
an accumulator that produces FSK mark and/or space threshold signals correlative to the multi-bit mark and/or multi-bit space values.

15. The device of claim 7 wherein the sample evaluation unit includes:
a filter coupled to the ADC;
an accumulator coupled to the filter, the filter and the accumulator demodulating a frequency shift keying (FSK) signal initially received at the coil unit; and
a deserializer that deserializes the demodulated FSK signal.

16. A device that is implantable within an individual's body, the device comprising:
a first circuit that receives communications via frequency shift keying (FSK) modulated time varying magnetic signals and converts the time varying magnetic signals into FSK modulated electrical signals;
a second circuit that samples the electrical signals, the second circuit having a sampling size of one bit; and
a third circuit that sums sampled bits of the second circuit to produce FSK mark and space recognition signals.

17. The device of claim 16, further comprising a fourth circuit that produces deserialized data using the FSK mark and space recognition signals.

18. A method for communicating with an implanted device within an individual's body, the method comprising:
producing a frequency modulated electrical signal based on a frequency modulated time varying magnetic signal;
sampling the electrical signal at periodic intervals, the sampled electrical signal having only bit that is associated with an individual periodic interval; and
summing individual sampled bits to produce one or more data recognition signals that discriminate between at least two frequencies of the electrical signal.

19. The method of claim 18 wherein the frequency modulate time varying signal is a frequency shift keying (FSK) modulated signal, and wherein the data recognition signals include FSK mark and space recognition signals.

20. The method of claim 18 wherein summing the individual sampled bits includes filtering the sampled electrical signal with a finite impulse response filter (FIR).

21. The method of claim 18 wherein summing the individual sampled bits is carried out, at least in part, using a leaky integrator.

22. The method of claim 18, further comprising using the data recognition signals to operate the implanted device, monitor the implanted device, and/or provide a medical treatment to the individual using the implanted device.

23. A method for communicating with an implanted device within an individual's body, the method comprising:
producing a time varying magnetic signal that is frequency shift keying (FSK) modulated; and
using the time varying magnetic signal to create an FSK modulated electrical signal at the implanted device, the electrical signal being communicated to a single-bit analog to digital converter (ADC) of the implanted device, and the single-bit ADC being used to at least partially demodulate the electrical signal.

24. The method of claim 23 wherein using the time varying magnetic signal further includes operating the implanted device, monitoring the implanted device, and/or providing a medical treatment to the individual using the implanted device.
